(19) United States
(12) Patent Application Publication
(10) Pub. No.: US 2007/0067043 A1
(43) Pub. Date: Mar. 22, 2007

(54) "Cement and bone graft absorbable & implantable detachable sac," a delivery system

(76) Inventor: Gerard Henry Dericks, Deming, NM (US)

Correspondence Address: Gerard Henry Dericks, Jr MD, 1311 Baird Court, Concord, CA, 94518, US

(21) Appl. No.: 11/122,304

(22) Filed: Sep. 19, 2005

(51) Int. Cl. A61F 2/28; A61B 17/88

(52) U.S. Cl. 623/23.52; 623/23.61; 606/93

(57) ABSTRACT

My proposed device is a fillable, detachable sac, which is intended to be placed with standard endoscopic or minimally invasive surgical techniques into bone cavities in Orthopedic Surgical practice. The “Sac” may be made of either permanent or dissolvable materials and is to be filled with bone cement, bone graft, antibiotic, or other pharmacologic materials as the particular pathology may dictate. Normally the “sac” or sack, will be introduced via an endoscopic cannula, and then, after filling, detached.

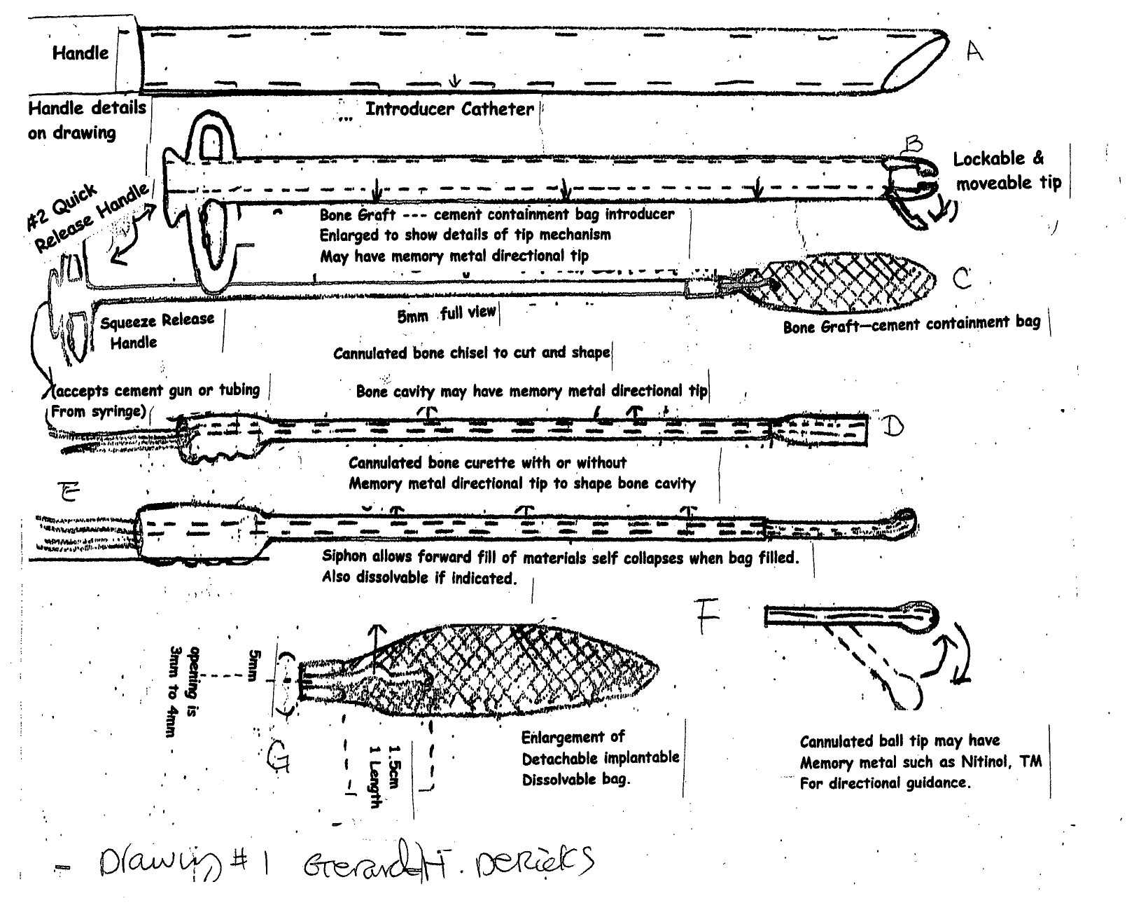

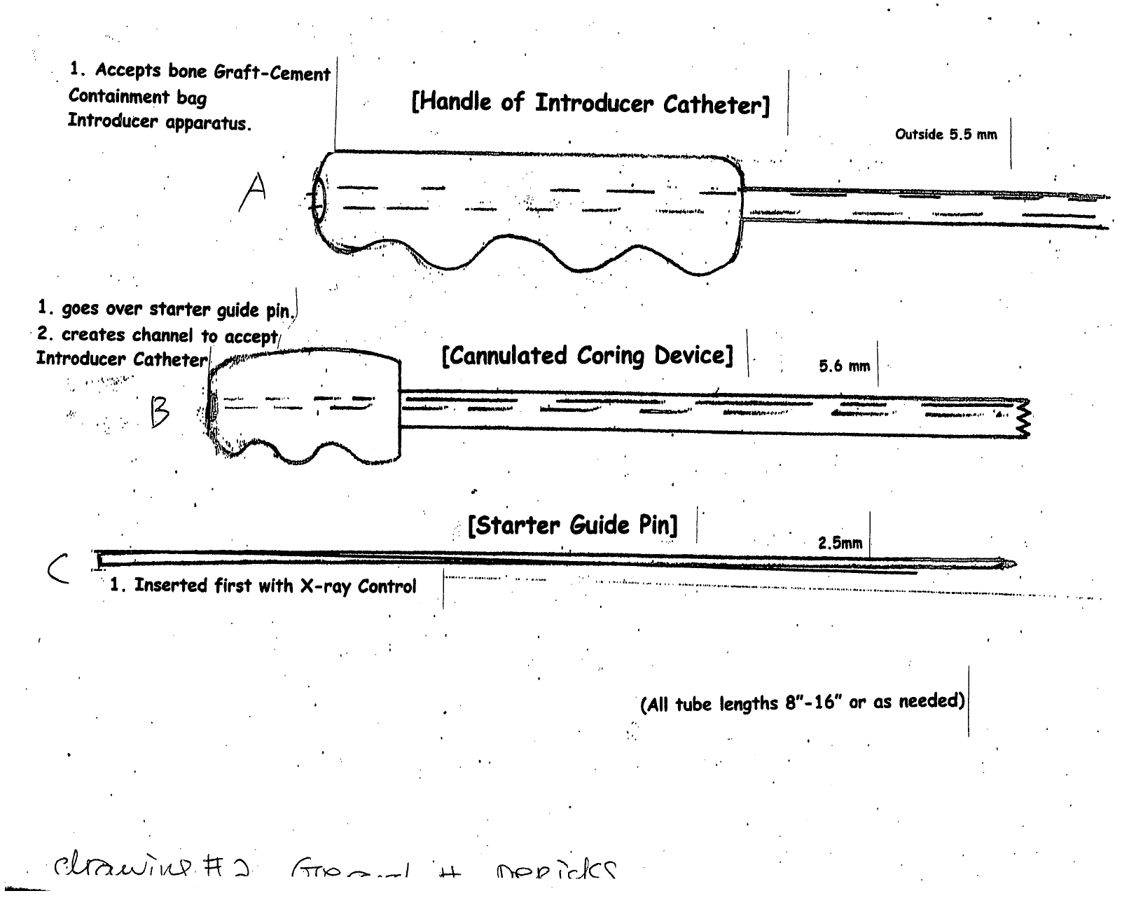

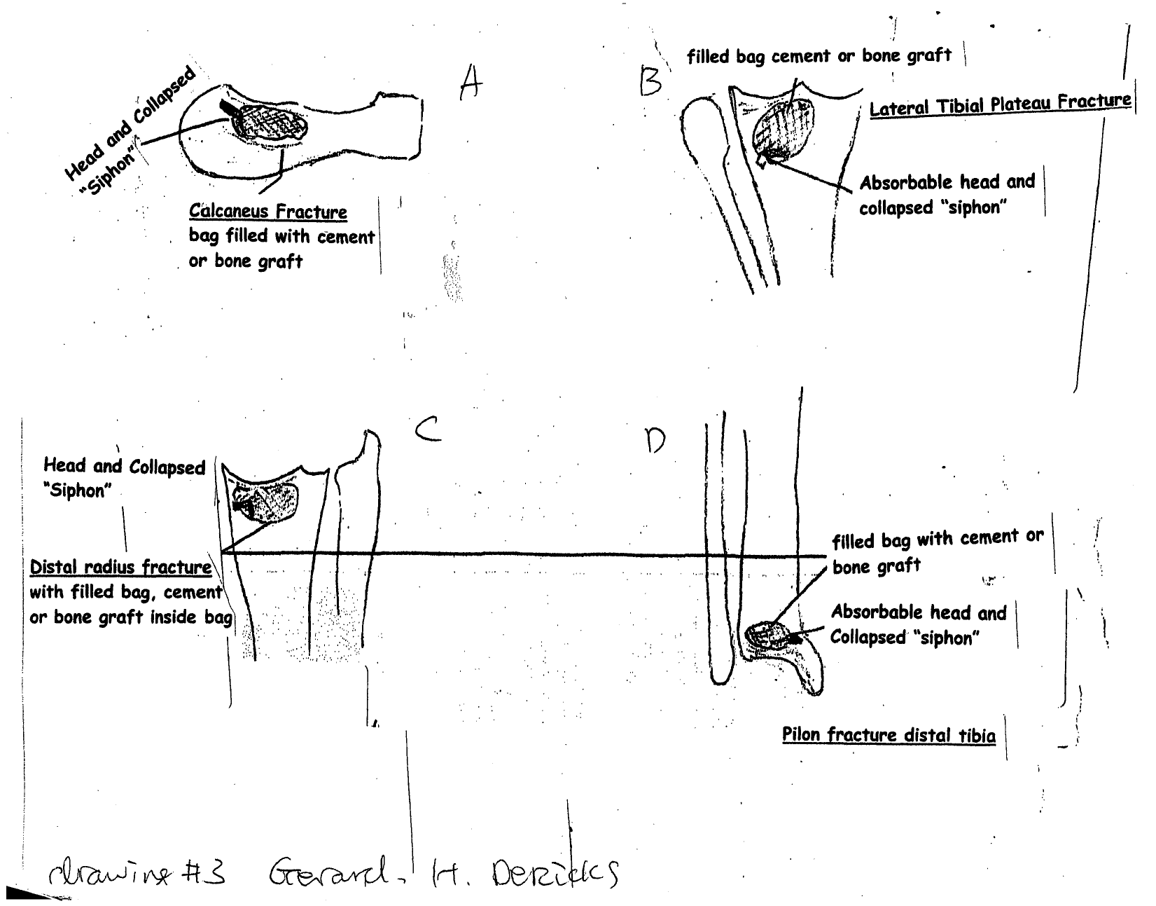

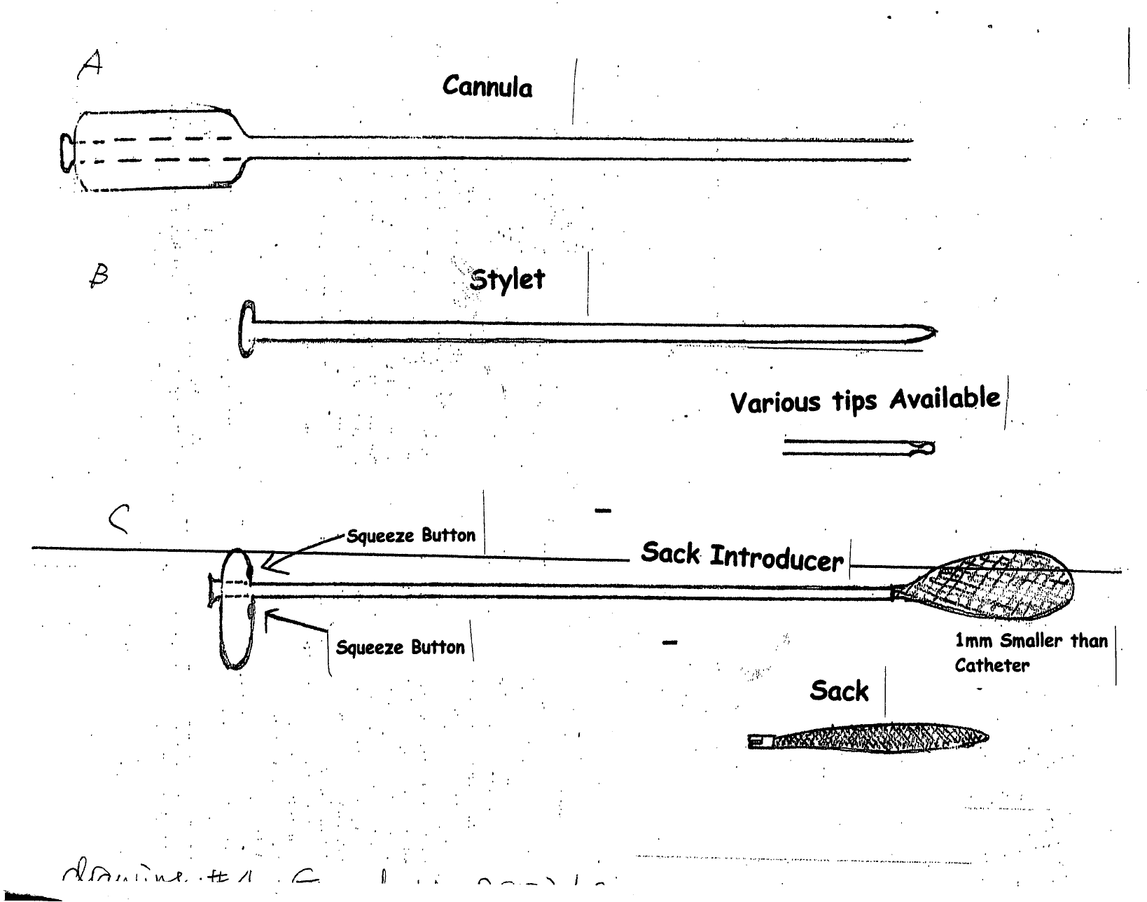

Instruments and devices have been created and patented to expand, compact and tamp cancellous bone to create a cavity in pathological or non pathological fractures, which bones then can be filled with various self-hardening cement or bone graft materials. Many of these fractures are close to vital structures (nerves, arteries, joint spaces, muscle groups, tendons etc.) and there exists a current problem with the containment of these grafting or fixating substances, which have great potential to create serious damage, if allowed to accidentally escape beyond the proposed grafting space.

This invention proposes an affordable, fillable, detachable and absorbable sac or sack, which can be placed down various sizes of catheters in common minimally-invasive surgical practice. The filling of the radioopaque (or non radioopaque) material can then be achieved without worry of leakage as “the sac” can be as completely impermeable (or not), as desired, to control precisely the delivery of these grafting or cementing substances (or other materials).

This device will greatly improve the safety of these grafting procedures which have been developed (and future such procedures) for areas of the body with cancellous bone such as vertebral bodies, distal radius calcaneal proximal and distal tibia, tarsal, carpal, navicular and radial head fractures, acetabular, pelvic and without limitation to any areas of the body where contained materials need to be delivered with bone graft, cement, drugs, chemotherapy, antibiotic or other medical devices impregnated cement or beads or self hardening bone grafts such as Norian—™. The absorbable, fillable, then detachable woven mesh “bag” can be made in various sizes from very small, such as 0.1 cc or smaller to very large 10-20 cc or larger. Cortical bone cementing or grafting techniques would be possible but are currently not feasible because without containment it is too dangerous.

It can be made of any approved existing man-made sugar material in a mesh pattern-such as in Polyglactin 910 Vicryl—™ Suture or PDS II—™ Polydioxanone and in variable thickness is for controlled absorption. It can also be made of natural absorbable materials similar to catgut or impermeable, semi permeable or even non absorbable materials such as Gortex—™ if desired or Dacron—™ or other polyester mesh, such as from LARS—™ or Ligastic materials.

By containing these potentially damaging substances, they can be delivered far more safely adjacent to joint spaces, vertebral end plates, spinal or other nerves, arteries, tendons and muscles. For example, a hard material such as self-hardening cement or bone paste, ordinary bone chips or ordinary bone paste could seriously damage the articular cartilage of an adjacent joint, or put undo pressure on adjacent nerves or vessels.

Bone cement while hardening gives off a great deal of heat which, if not safely contained as in my proposed device, could seriously damage spinal or other nerves, arteries, veins, muscles, tendons and joints (as they have delicate cartilaginous surfaces).

When the correct and desired filling and or consistency of the desired filler material has been achieved, the device can be simply unscrewed, unlocked or pulled apart at a crimp and the sac or bag can then dissolve (or not) at the desired, pre-determined rate, as predicted by the size, thickness and type of material chosen for that particular “bone containment sac”, in addition to precisely placed variable sized “holes” to create desired leakage in certain areas.

The detachable head of the sac can be radiopaque or lucent as desired. It will be made of hard dissolvable sugar or similar FDA approved material and have a “Luer-Lok”—™ attachment or a threaded or a quickly detachable mechanism built into or attached to the introducer shaft. The entire apparatus will fit down conventional catheters of varying sizes, in arthroscopic or endoslopic styles.

The “head” can be made from a U.S. FDA approved hard, dissolvable substance, with low allergenic potential, such as P.D.L.A. amorphous cupolymer as in the Arthrex Bio-Suture-Tak—™. This material is a non-crystalline copolymer, known as P.D.L.A. (L-Lactide-co-D, L-Lactide. It begins degradation at 12 weeks, completing in 16 months with total dissolution.

In the place of a conventional “Luer-Lok” or similar quick-coupling attachment, a crimp could be utilized (similar to the way “pop-off” needles are attached to some suture materials). Either way, a simple twisting action on the connecting shaft after filling of the sac or bag would assist in containment of the injected materials, which is then detached.

If a crimp is chosen, the sac could be detached by just pulling it apart after filling and hardening. If a Luer-Lok—™ type of attachment is chosen, the introducer shaft could be removed by twisting in one direction.

Current methods of filling these previously created cancellous bone defects are limited to techniques which involve differential fill rates to allow hardening in an egg shell pattern around a potentially dangerous location to check or reduce leakage first. Subsequent “backfilling” is then done, with complex unpredictable techniques being required to control delivery.

This unnecessary complexity of technique would be eliminated with the utilization of my proposed “bone graft (or cement) containment sac or bag.” This would result in considerable anesthesia and time and roentenographic, exposure time savings, benefiting the patient, entire operating room staff, anesthesiologist, and surgeon, as well as providing very significant cost and safety benefits. Expensive operating room time savings would also be achieved.

A unidirectional nitinol (or other memory metal or other “memory” material ball-tipped stylet is proposed to guide the “containment sac” to the defined location in the bone cavity prior to, or during the filling process.

Holes of varying sizes at the ends or sides of the implantable grafting “bag” will be built into different designs to move precisely control filling areas when desired. Cortical bone fracture grafting or fixation with self-hardening materials is also practical and possible. Cortical bone fractures may be reamed as necessary with flexible, expandable reaming systems

1. The invention claimed is a fillable detachable, absorbable (or non-absorbable) bone cement, pharmacological, or grafting materials containment “sac” or sack. It is designed to be implanted with commonly practiced minimally invasive or endoscopic techniques via a cannulated system. The use of such a device would provide a quantum advance in the safety of these grafting and cementing procedures.
